(19) United States
(12) Patent Application Publication
(10) Pub. No.: US 2009/0312296 A1
(43) Pub. Date: Dec. 17, 2009

(54) ACTIVE INGREDIENT COMBINATION OF A RETINOID AND A HORMONE COMBINATION WITH CONTRACEPTIVE ACTION AS MEDICAMENT FOR TREATMENT OF SKIN DISEASES

(75) Inventors: GEORG SCHRAMM, Stolberg (DE); KARL-HEINZ OEDEKOVEN, Stolberg (DE)

Correspondence Address: NORRIS, MCLAUGHLIN & MARCUS, PA, 875 THIRD AVENUE, 18TH FLOOR, NEW YORK, NY, 10022, US

(73) Assignee: GRUNENTHAL GMBH, Aachen (DE)

(21) Appl. No.: 12/483,857

(22) Filed: Jun. 12, 2009

Related U.S. Application Data
(63) Continuation of application No. PCT/EP2007/011143, filed on Dec. 19, 2007.

(30) Foreign Application Priority Data
Dec. 22, 2006 (DE) 10 2006 062 119.0

(51) Int. Cl. A61K 31/565; A61K 31/203; A61K 31/566; A61K 31/567; A61K 31/569; A61K 31/57; A61K 31/575; A61P 17/06; A61P 17/08; A61P 17/10; A61P 17/00; A61P 15/18

(52) U.S. Cl. 514/171; 514/560

(57) ABSTRACT

The present invention relates to a medicament, the active ingredient combination of which consists of a retinoid selected from the group consisting of acitretin [9-(4-methoxy-2,3,6-trimethylphenyl)-3,7-dimethylnona-2,4,6,8-tetraenoic acid], etretinate [ethyl 9-(4-methoxy-2,3,6-trimethylphenyl)-3,7-dimethylnona-2,4,6,8-tetraenoate], isotretinoin [3,7-dimethyl-9-(2,6,6-trimethyl-1-cyclohexen-1-yl)-2,4,6,8-nonatetraenoic acid] and tretinoin [3,7-dimethyl-9-(2,6,6-trimethyl-1-cyclohexenyl)nona-2,4,6,8-tetraenoic acid] and of a hormone combination with contraceptive action of an oestrogen component and a gestagen component, and to a dosage form consisting of at least 28 daily units, of which the final 7-3 daily units contain only the retinoid as active ingredient and the other daily units also contain the hormone-containing active ingredient combination, and to the use thereof for treating acne, seborrhoea or psoriasis.

The present invention relates to a medicament, the active ingredient combination of which consists of a retinoid selected from the group consisting of acitretin [9-(4-methoxy-2,3,6-trimethylphenyl)-3,7-dimethylnona-2,4,6,8-tetraenoic acid], etretinate [ethyl 9-(4-methoxy-2,3,6-trimethylphenyl)-3,7-dimethylnona-2,4,6,8-tetraenoate], isotretinoin [3,7-dimethyl-9-(2,6,6-trimethyl-1-cyclohexen-1-yl)-2,4,6,8-nonatetraenoic acid] and tretinoin [3,7-dimethyl-9-(2,6,6-trimethyl-1-cyclohexenyl)nona-2,4,6,8-tetraenoic acid] and of a hormone combination with contraceptive effect of an oestrogen component and a gestagen component, and to the use thereof as a dosage form for treating acne, seborrhoea or psoriasis.

Acne is an inflammatory disease of the sebaceous glands which primarily occurs during puberty. In its mild form, acne is a more or less superficial impairment which manifests as slight blotchy areas of irritation and can be sufficiently treated with skin cosmetics. In inflammatory forms of acne, however, bacteria penetrate into or around the hair follicles, so resulting in the formation of papules, pustules, nodules, infected pockets and in extreme cases infected cysts. Such inflammation may become extensive and form lasting scars.

At least 80% of teenagers are affected by acne. Facial rashes caused by acne may be a problem precisely during puberty because they impair the outward appearance of the developing person and in many cases may cause psychological disturbances, especially in girls. A therapeutic treatment for acne is therefore of extreme importance.

Retinoids have long been known. They are used as pharmaceutical active ingredients in medicines for treating skin diseases such as for example psoriasis or acne, in particular severe acne (cf. for example Mezick et al., J. Invest. Dermatol., 83:110-13, 1984). The retinoid isotretinoin (13-cis-retinoic acid), a cis-isomer of tretinoin, is accordingly used for producing a highly effective acne medicine which also exhibit numerous side-effects. As is the case with most orally administered retinoids, treatment with isotretinoin may possibly be associated with considerable risks, since this active ingredient exhibits numerous side-effects. Due to the side-effects, isotretinoin is thus usually only used to treat cases where the disease has taken a severe and treatment-resistant course.

One of most serious side-effects is a strong teratogenicity (teratogenic action) of the active ingredient, for which reason retinoids can only be taken by women of child-bearing age if there is no risk of pregnancy. For this reason, patients treated with retinoids are advised to avoid pregnancy during the period of treatment, preferably by taking contraceptives.

The object of the present invention was accordingly to provide a reliable contraception as effectively as possible during treatment with a retinoid-containing medicament.

Said object is achieved according to the invention by the provision of a medicament, the active ingredient combination of which consists of a retinoid used for acne treatment and of a hormone combination with contraceptive action of an oestrogen component and a gestagen component.

The present invention accordingly provides a medicament, the active ingredient combination of which consists of a retinoid selected from the group consisting of acitretin [9-(4-methoxy-2,3,6-trimethylphenyl)-3,7-dimethylnona-2,4,6,8-tetraenoic acid], etretinate [ethyl 9-(4-methoxy-2,3,6-trimethylphenyl)-3,7-dimethylnona-2,4,6,8-tetraenoate], isotretinoin [3,7-dimethyl-9-(2,6,6-trimethyl-1-cyclohexen-1-yl)-2,4,6,8-nonatetraenoic acid] and tretinoin [3,7-dimethyl-9-(2,6,6-trimethyl-1-cyclohexenyl)nona-2,4,6,8-tetraenoic acid] and of a hormone combination with contraceptive action of an oestrogen component and a gestagen component.

The medicament according to the invention ensures effective treatment while minimising the risk of pregnancy, since much better compliance with the therapeutic regimen is ensured.

Retinoids are chemical substances which are related in terms of their chemical structure or their biological activity with retinol (vitamin A). Selected retinoids are put to medical therapeutic use in humans. Three generations of therapeutically effective retinoids are known:
Non-aromatic retinoids (1st generation). Non-aromatic retinoids include tretinoin (all-trans-retinoic acid) and its isomer isotretinoin (13-cis-retinoic acid). Both also occur in comparatively small quantities in vivo as metabolites of vitamin A.
Mono-aromatic retinoids (2nd generation), for example acitretin, etretinate or motretinide,
Poly-aromatic retinoids (3rd generation), for example adapalene, arotinoid, acetylene retinoids or tazarotene.

Reference is made with regard to retinoid nomenclature to the publication “Nomenclature of retinoids” from the International Union of Pure and Applied Chemistry (IUPAC), Pure Appl Chem, 1983, V55(4), pp. 721-726.

Therapeutically effective retinoids which are present in the active ingredient combination according to the invention are 1st generation, 2nd generation or 3rd generation retinoids. The retinoid used is preferably at least one retinoid selected from the group consisting of acitretin [9-(4-methoxy-2,3,6-trimethylphenyl)-3,7-dimethylnona-2,4,6,8-tetraenoic acid], etretinate [ethyl 9-(4-methoxy-2,3,6-trimethylphenyl)-3,7-dimethylnona-2,4,6,8-tetraenoate], isotretinoin [3,7-dimethyl-9-(2,6,6-trimethyl-1-cyclohexen-1-yl)-2,4,6,8-nonatetraenoic acid] and tretinoin [3,7-dimethyl-9-(2,6,6-trimethyl-1-cyclohexenyl)nona-2,4,6,8-tetraenoic acid], particularly preferably isotretinoin [3,7-dimethyl-9-(2,6,6-trimethyl-1-cyclohexen-1-yl)-2,4,6,8-nonatetraenoic acid].

The oestrogen component for the hormone combination used is preferably at least one compound selected from the group consisting of oestradiol, oestrone, oestriol, oestradiol benzoate, oestradiol valerate, ethinyl oestradiol and ethinyl oestradiol-3-methyl ether. The oestrogen component present is particularly preferably ethinyl oestradiol or oestradiol and oestradiol is very particularly preferred, since this oestrogen, being a naturally occurring oestrogen, places a lower load on the liver.

The gestagen component of the hormone combination used according to the invention is preferably at least one compound selected from the group consisting of norethisterone, norethisterone acetate, norethisterone enantate, norgestimate, norgestrel, levonorgestrel, gestodene, hydroxyprogesterone caproate, medroxyprogesterone acetate, megestrol acetate, chlormadinone acetate, 3β-hydroxychlormadinone acetate (17α-acetoxychloropregna-4,6-dien-3β-ol-20-one), 3α-hydroxychlormadinone acetate (17α-acetoxychloropregna-4,6-dien-3α-ol-20-one), demegestone, lynestrenol, cyproterone acetate, drospirenone, dienogest, desogestrel, progesterone, dydrogesterone, medrogestone, promegestone, etonogestrel, norelgestromin, norethynodrel, nomegestrol acetate and trimegestone. Chlormadinone acetate, 3α-hydroxychlormadinone acetate and 3β-hydroxychlormadinone acetate are particularly preferred as the gestagen component.

An antiandrogenic gestagen for treating acne, seborrhoea or psoriasis is preferably used as the gestagen component in the medicament according to the invention.

The medicament according to the invention preferably comprises a specific number of daily units for oral administration. A specific number of these daily units containing the active ingredient combination are preferably combined to form a dosage form according to the invention for uninterrupted, daily oral administration in combination with a specific number of hormone-free daily units containing only the retinoid as active ingredient for immediately subsequent, uninterrupted, daily, oral administration to women.

The dosage form according to the invention preferably comprises at least 21-25 of the daily units containing the active ingredient combination and 7 to 3 hormone-free daily units containing only the retinoid as active ingredient.

The maximum number of daily units of a dosage form according to the invention may accordingly correspond to uninterrupted, daily, oral administration over 364 days, with 357 to 361 daily units containing the active ingredient combination for uninterrupted, daily, oral administration and 7 to 3 hormone-free daily units containing the retinoid for subsequent uninterrupted, daily, oral administration to women being present.

Table 1 summarises preferred explanations regarding the maximum number of daily units containing the active ingredient combination according to the invention or the hormone-free daily units of the dosage form according to the invention for uninterrupted administration.

TABLE 1

| | Number of daily | Number of hormone- |
|---|---|---|
| Duration of | units containing the | free daily units |
| uninterrupted | active ingredient | containing only |
| administration (days) | combination | retinoid |
| 364 | 357-351 | 7-3 |
| 336 | 329-333 | 7-3 |
| 308 | 301-305 | 7-3 |
| 280 | 273-277 | 7-3 |
| 252 | 245-249 | 7-3 |
| 224 | 217-221 | 7-3 |
| 196 | 189-193 | 7-3 |
| 168 | 161-165 | 7-3 |
| 140 | 133-137 | 7-3 |
| 112 | 105-109 | 7-3 |
| 84 | 77-81 | 7-3 |
| 56 | 49-53 | 7-3 |
| 28 | 21-25 | 7-3 |

A preferred dosage form is furthermore one whose maximum number of daily units is suitable for uninterrupted, daily, oral administration over 168 days, with 161 to 165 daily units containing the active ingredient combination for uninterrupted, daily, oral administration and 7 to 3 hormone-free daily units containing the retinoid for subsequent uninterrupted, daily, oral administration to women being present.

A likewise preferred dosage form is furthermore one whose maximum number of daily units is suitable for uninterrupted, daily, oral administration over 112 days, with 105 to 109 daily units containing the active ingredient combination for uninterrupted, daily, oral administration and 7 to 3 hormone-free daily units containing the retinoid for subsequent uninterrupted, daily, oral administration to women being present.

A likewise preferred dosage form is furthermore one whose maximum number of daily units is suitable for uninterrupted, daily, oral administration over 84 days, with 77 to 81 daily units containing the active ingredient combination according to the invention for uninterrupted, daily administration and 7 to 3 hormone-free daily units containing only the retinoid as active ingredient for subsequent uninterrupted, daily, oral administration to women being present.

A preferred embodiment of the dosage form according to the invention comprises a maximum number of daily units for uninterrupted, daily, oral administration for 28 days, with 21 to 25 daily units containing the active ingredient combination according to the invention for uninterrupted, daily administration and 7 to 3 hormone-free daily units containing only the retinoid as active ingredient for subsequent uninterrupted, daily, oral administration to women being present.

In a furthermore preferred embodiment, the dosage form according to the invention consists of up to 13 arrangements of in each case 28 daily units for uninterrupted, daily, oral administration, in each case 21 to 25 daily units containing the active ingredient combination for uninterrupted, daily administration being combined with in each case 7 to 3 hormone-free daily units containing only the retinoid as active ingredient for subsequent uninterrupted, daily, oral administration to women.

According to this preferred embodiment, the dosage form according to the invention may also consist of up to 6, preferably up to 4 of these arrangements of in each case 21 to 25 daily units containing the active ingredient combination for uninterrupted, daily oral administration combined with in each case 7 to 3 hormone-free daily units containing only the retinoid as active ingredient for subsequent uninterrupted, daily, oral administration to women.

Each daily unit containing the hormone-containing active ingredient combination preferably comprises in each case the same quantity of the gestagen component and in each case the same quantity of the oestrogen component.

Each daily unit containing the hormone-containing active ingredient combination of the dosage form according to the invention preferably contains the oestrogen component in each case in a quantity which stabilises the female menstrual cycle and the gestagen component in each case in a quantity with a contraceptive action. Stabilising quantities of the oestrogen component and quantities of the gestagen component with a contraceptive action are known to a person skilled in the art and summarised by way of example in Tables 2 and 3.

TABLE 2

| | Preferred quantity of active | |
|---|---|---|
| | ingredient per hormone- | |
| Oestrogen component | containing daily unit | |
| Oestradiol valerate | 0.5 to 4 | mg |
| Ethinyl oestradiol | 5 to 50 | μg |
| Mestranol | 8 to 100 | μg |
| Oestradiol | 0.5 to 5 | mg |
| Oestrone | 1 to 50 | mg |
| Oestriol | 1 to 50 | mg |
| Oestradiol benzoate | 0.5-4 | mg |

TABLE 3

| | Preferred quantity of active | |
|---|---|---|
| | ingredient per hormone- | |
| Gestagen component | containing daily unit | |
| Norethisterone, norethisterone acetate | 0.5 to 1.0 | mg |
| Norgestimate | 0.1 to 0.25 | mg |
| Norethisterone enantate | 1 to 3 | mg |
| Norgestrel | 0.3 to 1.0 | mg |
| Levonorgestrel | 0.05 to 0.15 | mg |
| Gestodene | 0.05 to 0.12 | mg |
| Hydroxyprogesterone caproate | 10 to 800 | mg |
| Medroxyprogesterone acetate | 2.5 to 40 | mg |
| Megestrol acetate | 1.0 to 10 | mg |
| Chlormadinone acetate | 0.5 to 10 | mg |
| 3α-Hydroxychlormadinone acetate | 1 to 20 | mg |
| 33-Hydroxychlormadinone acetate | 1 to 20 | mg |
| Lynestrenol | 0.4 to 3 | mg |
| Cyproterone acetate | 0.5 to 10 | mg |
| Drospirenone | 1.0 to 10 | mg |
| Dienogest | 1.0 to 10 | mg |
| Desogestrel | 0.06 to 0.30 | mg |
| Progesterone | 100 to 1000 | mg |
| Dydrogesterone | 5 to 50 | mg |
| Medrogestone | 2 to 30 | mg |
| Promegestone | 0.5 to 10 | mg |
| Nomegestrol acetate | 0.5 to 10 | mg |
| Trimegestone | 0.1 to 10 | mg |
| Etonogestrel | 0.1 to 1 | mg |
| Norelgestromin | 0.1 to 2 | mg |
| Norethynodrel | 0.3 to 3 | mg |
| Tibolone | 1 to 10 | mg |
| Demegestone | 0.1 to 10 | mg |

Each daily unit of the dosage form according to the invention preferably contains 1 to 100 mg, particularly preferably 1 to 75 mg and very particularly preferably 3 to 60 mg of retinoid, it possibly being necessary at the beginning of acne treatment to use the retinoid at a higher active ingredient dose than in the further course of treatment.

Each of the first 28 daily units provided for administration of a dosage form accordingly preferably in each case contains at least 30 mg, particularly preferably at least 50 mg of the retinoid and the subsequent, further daily units of the dosage form preferably in each case contain ½ to ⅓, particularly preferably ½ to ⅕ of the quantity of retinoid of the first 28 daily units provided for administration.

These 28 daily units provided for the beginning of administration preferably in each case comprise the same quantity of retinoid and the remaining daily units of the dosage form likewise in each case comprise the same quantity of retinoid.

The medicament according to the invention or the dosage form according to the invention is preferably suitable for preventing and/or treating acne. One of the major advantages of therapeutic treatment with the dosage form according to the invention is that acne treatment is not interrupted during the usual interval in taking of hormonal contraceptives and there are accordingly no crucial fluctuations in the patient's retinoid blood levels.

Acne is principally a disease of the sebaceous gland follicle, which initially causes non-inflammatory comedones but may subsequently also cause the occurrence of a range of inflammatory efflorescences (inter alia papules, pustules and nodules). In some forms of acne, terminal and vellus hair follicles may be affected.

Acne is classified depending on cause, form or severity and age.

The best known and most widespread form is acne vulgaris or “common acne”. “Common” in this context means that the acne first appears during puberty as a result of elevated androgen levels and subsides at the latest by the beginning of the 3rd decade of life. Because its causes lie in changes to the body's metabolism, acne vulgaris is one of the endogenous forms of acne.

If acne of presumably endogenous causation arises at an “uncommon” age, i.e. either prepubertally or in advanced adulthood (acne tarda), metabolic disorders typical thereof should be sought out.

Acne inversa is a frequently severe inflammation of the sebaceous glands and terminal hair follicles, preferably in intertriginous areas such as for example the armpits, groin region and gluteal fold.

Acne may occur not only in isolation but also in combination with other skin conditions, such as for example psoriasis, seborrhoea and/or rosacea, or one complaint can trigger and/or cause the other.

Seborrhoea is a medical term for pathologically changed sebaceous gland secretion.

It is primarily associated with androgens, relatively high androgen receptor density, androgen receptor sensitivity, with deficiency of biotin (vitamin B7) and vitamin B6 (pyridoxine). The term is sometimes also used for seborrhoeic eczema. Seborrhoea oleosa makes the skin oily, while seborrhoea sicca manifests itself in bran-like, greasy flakes. Seborrhoea encourages inter alia the occurrence of rosacea, acne vulgaris and acne necroticans.

Psoriasis is a skin disease, which is outwardly manifested substantially by a number of severely scaly, dot- to palm-sized patches of skin (often on the knees, elbows and the scalp). Psoriasis is a non-infectious autoimmune disease which, in addition to the skin, may also attack the joints (5-20%) and finger/toe nails.

The most commonly occurring form of psoriasis is Psoriasis vulgaris.

Another aspect of the present invention relates to the use of at least one retinoid and optionally a hormone combination with contraceptive action of an oestrogen component and a gestagen component for producing a dosage form in the form of the above-described daily units A and hormone-free daily units B for preventing and/or for treating acne, the acne preferably being selected from the group comprising acne aestivalis, acne aggregata, acne comedonica, acne conglobata, acne inversa, acne nodularis, acne papulopustulosa and acne vulgaris.

The dosage form according to the invention is likewise preferably also suitable for treating ichthyosis congenita, hyperkeratosis palmoplantaris, pustulosis palmoplantaris, lichen ruber planus of the skin and mucosa, pityriasis rubra pilaris and/or Darier's disease.

One preferred embodiment of the invention relates to the use of a retinoid selected from the group consisting of acitretin [9-(4-methoxy-2,3,6-trimethylphenyl)-3,7-dimethylnona-2,4,6,8-tetraenoic acid], etretinate [ethyl 9-(4-methoxy-2,3,6-trimethylphenyl)-3,7-dimethylnona-2,4,6,8-tetraenoate], isotretinoin [3,7-dimethyl-9-(2,6,6-trimethyl-1-cyclohexen-1-yl)-2,4,6,8-nonatetraenoic acid] and tretinoin [3,7-dimethyl-9-(2,6,6-trimethyl-1-cyclohexenyl)nona-2,4,6,8-tetraenoic acid], optionally an oestrogen component selected from the group consisting of oestradiol and ethinyl oestradiol and optionally a gestagen component selected from the group consisting of chlormadinone acetate, 3β-hydroxychlormadinone acetate (17α-acetoxychloropregna-4,6-dien-3β-ol-20-one) and 3α-hydroxychlormadinone acetate (17α-acetoxychloropregna-4,6-dien-3α-ol-20-one) for producing a dosage form in the form of the above-described daily units A and hormone-free daily units B for preventing and/or for treating acne.

It is very particularly preferred to use isotretinoin [3,7-dimethyl-9-(2,6,6-trimethyl-1-cyclohexen-1-yl)-2,4,6,8-nonatetraenoic acid], optionally an oestrogen component selected from the group consisting of oestradiol and ethinyl oestradiol and optionally a gestagen component selected from the group consisting of chlormadinone acetate, 3β-hydroxychlormadinone acetate (17α-acetoxychloropregna-4,6-dien-3β-ol-20-one) and 3α-hydroxychlormadinone acetate (17α-acetoxychloropregna-4,6-dien-3α-ol-20-one) for producing a dosage form in the form of the above-described daily units A and hormone-free daily units B for preventing and/or for treating acne, seborrhoea, psoriasis and/or rosacea, particularly preferably for treating acne. The dosage form preferably comprises the daily units in the form of tablets which, in addition to the above-stated active ingredient or the above-stated active ingredient combination, preferably comprise conventional auxiliary substances.

The following examples serve to explain the invention further but should not to be construed as limiting the scope of the invention:

### EXAMPLE 1

### Composition

| | Per tablet | |
|---|---|---|
| Isotretinoin | 20 | mg |
| Ethinyl oestradiol | 0.020 | mg |
| Chlormadinone acetate | 2.000 | mg |
| Povidone K30 | 3.000 | mg |
| Lactose | 31.980 | mg |
| Maize starch | 12.000 | mg |
| Magnesium stearate | 0.500 | mg |
| Highly disperse silicon dioxide | 0.500 | mg |

The isotretinoin (ISO), ethinyl oestradiol (EO) and povidone K 30 (polyvinylpyrrolidone) are dissolved in 600 ml of ethanol. The chlormadinone acetate (particle size 90%<50 μm), lactose and maize starch are mixed for 5 min in a mixer/granulator (Diosna P25) and then thoroughly moistened and mixed with the ethanolic ISO/EO/PVP solution. The moist composition is forced through a 3 mm screen and dried in a vacuum drying cabinet. The dried granular product is disagglomerated through a 0.6 mm screen, mixed with magnesium stearate and highly disperse silicon dioxide and pressed on a tablet press with 5 mm punches into tablets with a weight of 70 mg. The hormone-free tablets comprise 34 mg of lactose.

The tablets are coated with a lacquer based on methylhydroxypropylcellulose (for example Opadry YS-1-2184 manufactured by Colorcon); coating mass 2 mg per tablet. The dosage form comprises 364 daily units, the final 7 of which are hormone-free, for uninterrupted administration.

### EXAMPLE 2

### Composition

| | Per tablet | |
|---|---|---|
| Isotretinoin | 20 | mg |
| Oestradiol | 1 | mg |
| Chlormadinone acetate | 2.000 | mg |
| Povidone K30 | 3.000 | mg |
| Lactose | 31 | mg |
| Maize starch | 12.000 | mg |
| Magnesium stearate | 0.500 | mg |
| Highly disperse silicon dioxide | 0.500 | mg |

The isotretinoin (ISO), oestradiol (OD) and povidone K 30 (polyvinylpyrrolidone) are dissolved in 600 ml of ethanol. The chlormadinone acetate (particle size 90%<50 μm), lactose and maize starch are mixed for 5 min in a mixer/granulator (Diosna P25) and then thoroughly moistened and mixed with the ethanolic ISO/OD/PVP solution. The moist composition is forced through a 3 mm screen and dried in a vacuum drying cabinet. The dried granular product is disagglomerated through a 0.6 mm screen, mixed with magnesium stearate and highly disperse silicon dioxide and pressed on a tablet press with 5 mm punches into tablets with a weight of 70 mg. The hormone-free tablets contain 34 mg of lactose.

The tablets are coated with a lacquer based on methylhydroxypropylcellulose (for example Opadry YS-1-2184 manufactured by Colorcon); coating mass 2 mg per tablet. The dosage form consists of 364 daily units, the final 7 of which are hormone-free, for uninterrupted administration.

### EXAMPLE 3

### Composition

| | Per tablet | |
|---|---|---|
| Isotretinoin | 40 | mg |
| Ethinyl oestradiol | 0.015 | mg |
| Chlormadinone acetate | 2.000 | mg |
| Povidone K30 | 4.000 | mg |
| Lactose | 43.485 | mg |
| Maize starch | 10.000 | mg |
| Magnesium stearate | 0.500 | mg |

The isotretinoin (ISO), ethinyl oestradiol (EO) and povidone K 30 (PVP) are dissolved in 950 ml of ethanol. The chlormadinone acetate (particle size 90%<50 μm), lactose and maize starch are mixed for 5 min in a mixer/granulator (Diosna P25) and then thoroughly moistened and mixed with the ethanolic ISO/EO/PVP solution. The moist composition is forced through a 3 mm screen and dried in a vacuum drying cabinet. The dried granular product is disagglomerated through a 0.6 mm screen, mixed with magnesium stearate and pressed on a tablet press with 6 mm punches into tablets with a weight of 100 mg. The hormone-free tablets contain 45.5 mg of lactose.

The tablets are coated with a methylhydroxypropylcellulose-based lacquer of the following composition (coating mass 2 mg per tablet)

| | |
|---|---|
| Methylhydroxypropylcellulose, 6 mPa · s | 0.1351 kg |
| Polyethylene glycol 6000 | 0.0395 kg |
| Propylene glycol | 0.0054 kg |
| Purified water | 1.6200 kg |

The dosage form contains 196 daily units, the final 7 of which are hormone-free, for uninterrupted administration.

1-25. (canceled)

26. A dosage form comprising a specific number of daily units for uninterrupted, daily oral administration, the active ingredient combination comprising of at least one retinoid selected from the group consisting of acitretin [9-(4-methoxy-2,3,6-trimethylphenyl)-3,7-dimethylnona-2,4,6,8-tetraenoic acid], etretinate [ethyl 9-(4-methoxy-2,3,6-trimethylphenyl)-3,7-dimethylnona-2,4,6,8-tetraenoate], isotretinoin [3,7-dimethyl-9-(2,6,6-trimethyl-1-cyclohexen-1-yl)-2,4,6,8-nonatetraenoic acid] and tretinoin [3,7-dimethyl-9-(2,6,6-trimethyl-1-cyclohexenyl)nona-2,4,6,8-tetraenoic acid] and of a hormone combination with contraceptive effect of at least one oestrogen component and a gestagen component, and which is optionally combined with a specific number of hormone-free daily units containing only the retinoid as active ingredient for immediately subsequent, uninterrupted, daily oral administration to women.

27. A dosage form according to claim 26, wherein isotretinoin [3,7-dimethyl-9-(2,6,6-trimethyl-1-cyclohexen-1-yl)-2,4,6,8-nonatetraenoic acid] is present as the retinoid.

28. A dosage form according to claim 26, wherein the oestrogen component is selected from the group consisting of oestradiol, oestrone, oestriol, oestradiol benzoate, oestradiol valerate, ethinyl oestradiol and ethinyl oestradiol-3-methyl ether.

29. A dosage form according to claim 28, wherein the oestrogen component is ethinyl oestradiol or oestradiol.

30. A dosage form according to claim 26, wherein the gestagen component is selected from the group consisting of norethisterone, norethisterone acetate, norethisterone enantate, norgestimate, norgestrel, levonorgestrel, gestodene, hydroxyprogesterone caproate, medroxyprogesterone acetate, megestrol acetate, chlormadinone acetate, 3β-hydroxychlormadinone acetate (17α-acetoxychloropregna-4,6-dien-3β-ol-20-one), 3α-hydroxychlormadinone acetate (17α-acetoxychloropregna-4,6-dien-3α-ol-20-one), demegestone, lynestrenol, cyproterone acetate, drospirenone, dienogest, desogestrel, progesterone, dydrogesterone, medrogestone, promegestone, etonogestrel, norelgestromin, norethynodrel, nomegestrol acetate and trimegestone.

31. A dosage form according to claim 30, wherein the gestagen component is at least one antiandrogenic.

32. A dosage form according to claim 31, wherein the gestagen component is selected from the group consisting of hydroxyprogesterone caproate, chlormadinone acetate, cyproterone acetate, drospirenone, dienogest, progesterone, dydrogesterone, promegestone, nomegestrol acetate and trimegestone.

33. A dosage form according to claim 30, wherein the gestagen component is selected from the group chlormadinone acetate, 3β-hydroxychlormadinone acetate (17α-acetoxychloropregna-4,6-dien-3β-ol-20-one) and 3α-hydroxychlormadinone acetate (17α-acetoxychloropregna-4,6-dien-3α-ol-20-one).

34. A dosage form according to claim 26, wherein the dosage form comprises at least 21 daily units containing the active ingredient combination and 7 to 3 hormone-free daily units containing only the retinoid as active ingredient.

35. A dosage form according to claim 26, wherein the maximum number of daily units of the dosage form corresponds to uninterrupted, daily, oral administration for 364 days, with 357 to 361 daily units containing the active ingredient combination for uninterrupted, daily, oral administration and 7 to 3 hormone-free daily units containing only the retinoid as active ingredient for subsequent uninterrupted, daily, oral administration to women being present.

36. A dosage form according to claim 26, wherein the maximum number of daily units of the dosage form corresponds to uninterrupted, daily, oral administration for 196 days, with 189 to 193 daily units containing the active ingredient combination for uninterrupted, daily, oral administration and 7 to 3 hormone-free daily units containing only the retinoid as active ingredient for subsequent uninterrupted, daily, oral administration to women being present.

37. A dosage form according to claim 26 wherein the maximum number of daily units of the dosage form corresponds to uninterrupted, daily, oral administration for 168 days, with 161 to 165 daily units containing the active ingredient combination for uninterrupted, daily, oral administration and 7 to 3 hormone-free daily units containing only the retinoid as active ingredient for subsequent uninterrupted, daily, oral administration to women being present.

38. A dosage form according to claim 26, wherein the maximum number of daily units of the dosage form corresponds to uninterrupted, daily, oral administration for 112 days, with 105 to 109 daily units containing the active ingredient combination for uninterrupted, daily, oral administration and 7 to 3 hormone-free daily units containing only the retinoid as active ingredient for subsequent uninterrupted, daily, oral administration to women being present.

39. A dosage form according to claim 26, wherein the maximum number of daily units of the dosage form corresponds to uninterrupted, daily, oral administration for 84 days, with 77 to 81 daily units containing the active ingredient combination for uninterrupted, daily, oral administration and 7 to 3 hormone-free daily units containing the retinoid for subsequent uninterrupted, daily, oral administration to women being present.

40. A dosage form according to claim 26, wherein the dosage form comprises 21 to 25 daily units containing the active ingredient combination for uninterrupted, daily, oral administration and 7 to 3 hormone-free daily units containing only the retinoid as active ingredient for subsequent, daily, oral administration to women.

41. A dosage form according to claim 40, wherein the dosage form comprises of at most 13 arrangements, each arrangement comprising 21 to 25 daily units containing the active ingredient combination provided for uninterrupted, daily, oral administration and each 7 to 3 hormone-free daily units containing only the retinoid as active ingredient provided for immediately subsequent uninterrupted, daily, oral administration to women.

42. A dosage form according to claim 26, wherein each daily unit containing the hormone-containing active ingredient combination contains the oestrogen component in each case in a quantity which stabilises the menstrual cycle and the gestagen component in each case in a quantity with a contraceptive action.

43. A dosage form according to claim 26, wherein each of the daily units containing the hormone-containing active ingredient combination contains the same quantity of the gestagen component and the same quantity of the oestrogen component.

44. A dosage form according to claim 26, wherein each daily unit of the dosage form contains 1 to 75 mg of the retinoid.

45. A dosage form according to claim 44, wherein 28 daily units of a dosage form provided at the beginning of administration contain at least 10 mg of the retinoid and the remaining daily units of the dosage form contain only 2 to 5 mg of retinoid.

46. A dosage form according to claim 45, wherein the 28 daily units of a dosage form provided at the beginning of administration comprise the same daily quantity of the retinoid and the remaining daily units of the dosage form comprise the same daily quantity of the retinoid, which is reduced relative to the 28 daily units administered at the beginning.

47. A dosage form according to claim 44, wherein each daily unit of the dosage form contains 3 to 30 mg of retinoid.
